(19) United States
(12) Patent Application Publication
(10) Pub. No.: US 2007/0088023 A1
(43) Pub. Date: Apr. 19, 2007

(54) Method of administration for 5-(2-chlorophenyl)-1,2-dihydro-7-fluoro-8-methoxy-3-methyl-oyrazolo[1,4]benzodiazepine

(76) Inventor: Stanislaw M. Mikulski, Essex Fells, NJ (US)

Correspondence Address: HOFFMANN-LA ROCHE INC.;PATENT LAW DEPARTMENT, 340 KINGSLAND STREET, NUTLEY, NJ, 07110, US

(21) Appl. No.: 11/544,838

(22) Filed: Oct. 6, 2006

Related U.S. Application Data
(60) Provisional application No. 60/727,020, filed on Oct. 14, 2005.

(51) Int. Cl. A61K 31/551

(52) U.S. Cl. 514/220

(57) ABSTRACT

The invention relates to improved methods of administration of 5-(2-chlorophenyl)-1, 2-dihydro-7-fluoro-8-methoxy-3-methyl-pyrazolo[3,4-b][1,4]benzodiazepine in the treatment of cancer, such as breast, colon, lung, and prostate cancer. In particular, the invention relates to a method of treating cancer by administering the above compound or a therapeutically effective salt or ester thereof in an amount from about 1.5 mg/m²/day to 30 mg/m²/day for a period of up to about 14 days every 3 weeks.

### PRIORITY TO RELATED APPLICATION

This application claims the benefit of U.S. Provisional Application No. 60/727,020, filed Oct. 14, 2005, which is hereby incorporated by reference in its entirety.

### FIELD OF THE INVENTION

The present invention is directed to improved methods of administration of 5-(2-chlorophenyl)-1,2-dihydro-7-fluoro-8-methoxy-3-methyl-pyrazolo[3,4-b][1,4] benzodiazepine in the treatment of cancer. In particular, the invention is directed to improved methods of administration of 5-(2-chlorophenyl)-1,2-dihydro-7-fluoro-8-methoxy-3-methyl-pyrazolo[3,4-b][1,4]benzodiazepine that provide desirable antineoplastic effects with a tolerable level toxicity.

### BACKGROUND OF THE INVENTION

The compound, 5-(2-chlorophenyl)-1,2-dihydro- 7-fluoro-8-methoxy-3-methyl-pyrazolo[3,4-b][1,4]benzodiazepine, having the structural formula 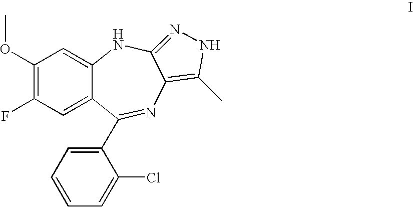
is an inhibitor of angiogenesis via inhibition of growth factor receptor tyrosine kinases, i.e., VEGF-R2, FGFR and PDGFR and kinases, such as cyclin-dependent kinases (CDKs), in particular Aurora A and CDK2. The compound and its pharmaceutically acceptable salts, and the esters of said compound, are anti-proliferative agents useful in the treatment or control of cell proliferative disorders, in particular cancer. The compound of the invention is especially useful in the treatment or control of breast, colon, lung and prostate tumors. The above compound is described in commonly owned U.S. application Ser. No. 11/244,251, incorporated by reference herein. The above compound is especially effective, and best tolerated, in cancer therapy when administered in specific doses and pursuant to the specific protocols herein described.

### SUMMARY OF THE INVENTION

The present invention relates to a method of treating a patient suffering from cancer comprising administering to the patient the compound of formula I, or a therapeutically effective salt or ester thereof, in an amount from about 1.5 mg/m²/day to 30 mg/m²/day, for an administration period of up to about 14 days every 3 weeks.

### DETAILED DESCRIPTION OF THE INVENTION

The present invention relates to a method of treating a patient having cancer which comprises administering to the patient a compound of the formula 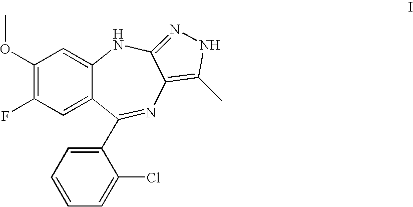
or a therapeutically effective salt or ester thereof in an amount from about 1.5 mg/m²/day to about 30 mg/m²/day for an administration period of up to 14 days every 3 weeks.

A preferred dosage regimen is 1.5 mg/m²/day given for a 14 day period.

Another preferred dosage regimen is 3 mg/m²/day given for a 14 day period.

Yet another preferred dosage regimen is 4.5 mg/m²/day given for a 14 day period.

Another preferred dosage regimen is 6 mg/m²/day given for a 14 day period.

Yet another preferred dosage regimen is 7.5 mg/m²/day given for a 14 day period.

Another preferred dosage regimen is 9 mg/m²/day given for a 14 day period.

Yet another preferred dosage regimen is 10.5 mg/m²/day given for a 14 day period.

Another preferred dosage regimen is 12 mg/m²/day given for a 14 day period.

Yet another preferred dosage regimen is 13.5 mg/m²/day given for a 14 day period.

Another preferred dosage regimen is 15 mg/m²/day given for a 14 day period.

Yet another preferred dosage regimen is 16.5 mg/m²/day given for a 14 day period.

Another preferred dosage regimen is 18 mg/m²/day given for a 14 day period.

Yet another preferred dosage regimen is 19.5 mg/m²/day given for a 14 day period.

Another preferred dosage regimen is 21 mg/m²/day given for a 14 day period.

Yet another preferred dosage regimen is 22.5 mg/m²/day given for a 14 day period.

Another preferred dosage regimen is 24 mg/m²/day given for a 14 day period.

Yet another preferred dosage regimen is 25.5 mg/m²/day given for a 14 day period.

Another preferred dosage regimen is 27 mg/m²/day given for a 14 day period.

Yet another preferred dosage regimen is 28.5 mg/m²/day given for a 14 day period.

Another preferred dosage regimen is 30 mg/m²/day given for a 14 day period.

The compound is provided as a tablet which is film coated using commercially available Opadry® which is a hydroxypropyl methylcellulose based coating system. Hydroxypropyl methylcellulose is used as a binder, Croscarmellose Sodium is used as a disintegrant, lactose hydrous as a diluent and magnesium stearate as a lubricant. The tablets are supplied as 1 mg, 5 mg and 20 mg tablets packed in vials.

The dose to be administered is calculated using body surface per m² rounded to the nearest practical dose using the tablet strengths described above.

“Therapeutically effective salt” refers to conventional acid-addition salts or base-addition salts which retain the biological effectiveness and properties of the compounds of formula IV and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium and, quaternary ammonium hydroxides, such as for example, tetramethylammonium hydroxide.

The term “therapeutically effective esters” embraces derivatives of the compounds of formula (I), in which a carboxy group has been converted to an ester. Lower-alkyl, hydroxy-lower-alkyl, lower-alkoxy-lower-alkyl, amino-lower-alkyl, mono- or di-lower-alkyl-amino-lower-alkyl, morpholino-lower-alkyl, pyrrolidino-lower-alkyl, piperidino-lower-alkyl, piperazino-lower-alkyl, lower-alkyl-piperazino-lower-alkyl and aralkyl esters are examples of suitable esters. The methyl, ethyl, propyl, butyl and benzyl esters are preferred esters. The methyl and ethyl esters are especially preferred. The term “therapeutically effective esters” furthermore embraces compounds of formula (I) in which hydroxy groups have been converted to the corresponding esters with inorganic or organic acids such as, nitric acid, sulphuric acid, phosphoric acid, citric acid, formic acid, maleic acid, acetic acid, succinic acid, tartaric acid, methanesulphonic acid, p-toluenesulphonic acid and the like, which are non toxic to living organisms.

The term “therapeutically effective amount” means an amount of drug, or combination or composition, which is effective for producing a desired therapeutic effect upon administration to a patient, for example, to stem the growth, or result in the shrinkage, of a cancerous tumor.

A patient's body measurement in square meters (“m²”) is a “BSA (body surface area) measurement”, typically ranges from about 1.4 m² to about 2.2. m². Thus, the total amount of the compound of Formula I to be delivered in a treatment cycle (mg) is calculated as follows:
[Dose intensity(mg/m²/week)]×[BSA(m²)]×[number of weeks in treatment cycle]

### EXPERIMENTAL

An open label, multi-center, multiple ascending dose study of the compound of Formula I as a single agent, administered orally on a daily×14 days, every 3 weeks schedule has begun. A completion of one 3-week cycle of treatment will be the basis for determining the maximum tolerated dose (MTD) on this schedule.

The starting dose is based on pre-clinical good laboratory practices toxicological results, according to the accepted standards. The pre-clinical toxicology data shows that the maximum tolerated dose in rats is 3 mg/kg/day times 6 which equals 18/mg/kg/day as a HED (human equivalent dose). Thus, the maximum tolerated dose equivalent for this trial will be 1/10^{th} of the HED or 1.8 rounded down to 1.5 mg/m²/day with dose escalation in 1.5 mg/m² increments to 30 mg/m² or until dose limiting toxicity(s)(DLT) occur.

Following determination of eligibility, patients are orally administered the compound of Formula I once daily for 14 consecutive days over a period of 3 week schedule. The compound of Formula I is administered at ascending dose levels on a schedule as defined above. One 3-week cycle is considered the treatment interval for determination of DLT (Dose limiting Toxicity) and MTD (maximum tolerated dose).

A minimum of 3 patients per cohort are enrolled. In each cohort. Initially one patient is initially treated and observed at least for 21 days. If no DLT occurs in the first patient, then two additional patients are treated at the same dose level and observed for 21 days. If 1 patient out of 3 experiences DLT, then the cohort is expanded to 6 patients. The recommended Phase II dose is one level below the dose at which 2 out of 6 patients experience DLT.

The first DLT which occurs during the first 3-week cycle of treatment will prompt expansion of that dose level to a minimum of 6 patients. After an occurrence of DLT, all subsequent cohorts will be expanded a priori to a minimum of 6 patients. If no further DLT occurs in any other patient in the expanded cohort (i.e., only 1 of 6 patients develops DLT), then dose escalation will proceed to the next level. If ≧2 of 6 patients in the expanded cohort develop DLT during their first treatment cycle, then the treatment at that dose level will be stopped, and the preceding dose level cohort will be expanded to 6 patients, if this has not already occurred. The highest dose level at which no more than 1 out of 6 patients experience a DLT will be considered the MTD and the recommended Phase II dose.

Dose escalation will be by 100% increments until Grade 2 drug-related toxicity occurs (according to NCI-CTCAE version 3.0). Subsequently, 50% dose escalation increments will be used until the first DLT (toxicity Grade≧ 3) is observed. If the first DLT is observed during the 50% escalation increments, the dose escalation will then be reduced to 25% of the preceding dose level.

Dose-Limiting Toxicity (DLT) is assessed during the first treatment cycle, and is defined as:
Any non-hematologic toxicity≧Grade 3 according to NCI-CTCAE version 3.0, except for selected cardiac toxicities as defined below. Nausea/vomiting, and/or diarrhea will be considered DLT only if they reach ≧Grade 3 severity despite adequate supportive care measures.
Grade 4 neutropenia lasting at least 7 days.
Febrile neutropenia (ANC <1.0×10⁹/L and fever ≧38.5° C.), and/or documented infection with ANC <1.0×10⁹/L.
Thrombocytopenia Grade 3 (i.e.,<25.0×10⁹/L according to the NCI-CTCAE version 3.0), or any thrombocytopenia requiring platelet transfusion.
Delay of treatment for >14 days for Cycle 2-Day 1.
Any of the following cardiac toxicities:
   New onset of conduction abnormality such as, atrioventricular block requiring medical intervention;
   New onset of cardiac arrhythmia requiring medical intervention, except atrial fibrillation Grade 2;
   New onset of symptomatic or asymptomatic cardiac ischemia;
   cTnT (Cardiac Troponin T)≧0.08 ng/mL ( in the face of adequate renal function);
   20% decrease in LVEF (Left Ventricular Ejection Fraction) when compared to baseline, if final EF is ≧50%;
   Any decrease in LVEF when compared to baseline, if final EF is <50%.

What is claimed:

1. A method of treating cancer in a patient in need thereof with a regimen comprising dosing a compound of the formula 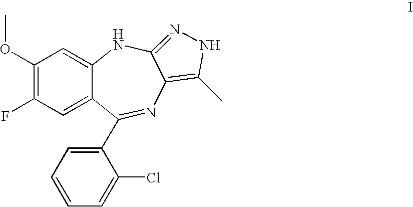
or a therapeutically effective salt or ester thereof in a dosage amount of from about 1.5 mg/m²/day to about 30 mg/m²/day for an administration period of up to 14 days every 3 weeks.

2. The method of claim 1 wherein the dosage amount is 1.5 mg/m²/day given for a 14 day period every 3 weeks.

3. The method of claim 1 wherein the dosage amount is 3 mg/m²/day given for a 14 day period every 3 weeks.

4. The method of claim 1 wherein the dosage amount is 4.5 mg/m²/day given for a 14 day period every 3 weeks.

5. The method of claim 1 wherein the dosage amount is 6 mg/m²/day given for a 14 day period every 3 weeks.

6. The method of claim 1 wherein the dosage amount is 7.5 mg/m²/day given for a 14 day period every 3 weeks.

7. The method of claim 1 wherein the dosage amount is 9 mg/m²/day given for a 14 day period every 3 weeks.

8. The method of claim 1 wherein the dosage amount is 10.5 mg/m²/day given for a 14 day period every 3 weeks.

9. The method of claim 1 wherein the dosage amount is 12 mg/m²/day given for a 14 day period every 3 weeks.

10. The method of claim 1 wherein the dosage amount is 13.5 mg/m²/day given for a 14 day period every 3 weeks.

11. The method of claim 1 wherein the dosage amount is 15 mg/m²/day given for a 14 day period every 3 weeks.

12. The method of claim 1 wherein the dosage amount is 16.5 mg/m²/day given for a 14 day period every 3 weeks.

13. The method of claim 1 wherein the dosage regimen is 18 mg/m²/day given for a 14 day period every 3 weeks.

14. The method of claim 1 wherein the dosage regimen is 19.5 mg/m²/day given for a 14 day period every 3 weeks.

15. The method of claim 1 wherein the dosage regimen is 21 mg/m²/day given for a 14 day period every 3 weeks.

16. The method of claim 1 wherein the dosage regimen is 22.5 mg/m²/day given for a 14 day period every 3 weeks.

17. The method of claim 1 wherein the dosage regimen is 24 mg/m²/day given for a 14 day period every 3 weeks.

18. The method of claim 1 wherein the dosage regimen is 25.5 mg/m²/day given for a 14 day period every 3 weeks.

19. The method of claim 1 wherein the dosage regimen is 27 mg/m²/day given for a 14 day period every 3 weeks.

20. The method of claim 1 wherein the dosage regimen is 28.5 mg/m²/day given for a 14 day period every 3 weeks.

21. The method of claim 1 wherein the dosage regimen is 30 mg/m²/day given for a 14 day period every 3 weeks.

22. The method of claim 1 wherein the cancer is breast cancer.

23. The method of claim 1 wherein the cancer is colon cancer.

24. The method of claim 1 wherein the cancer is lung cancer.

25. The method of claim 1 wherein the cancer is prostate cancer.
